SEQUENCE LISTING (32 sequences)

SEQ ID NO: 1 - protein, 121 aa, synthetic construct
QVQLQQSGAELARPGASVKLSCKASGYTFTSYGLSWVKQRTGQGLEWIGEIFPRSGNSYYNEKFKGKAALTADKSSSTAYMQLSSLTSEDSAVYFCARHYYSSLYYAMDYWGQGTSVTVSS

SEQ ID NO: 2 - protein, 107 aa, synthetic construct
DIQVTQSPASLSASVGESVSITCGTNENLYGALNWYQRKQGKSPQLLIYGATNLADGMSSRFSGSGSGRQYSLKISSLHPDDVATYYCQNVLSTPRTFGGGTKLEIK

SEQ ID NO: 3 - protein, 117 aa, synthetic construct
EVQLVESGGGLVKPGGSLKLSCAASGFSFSDYYMYWVRQAPEKRLEWVASISFDGTYTYYTDRVKGRFTISRDNAKNNLYLQMSSLKSEDTAMYYCARDRPAWFPYWGQGTLVTVSA

SEQ ID NO: 4 - protein, 112 aa, synthetic construct
DVLMTQIPLSLTVSLGDQASISCRSSQSIVHSNGNTYLEWYLQRPGQSPKLLIYKVSNRFSGVPDRFSGSGSGTDFTLKISRVEAEDLGVYYCFQGSHVPLTFGAGTKLELK

SEQ ID NO: 5 - protein, 119 aa, synthetic construct
EVKLQESGPELVKPGASVTISCKASGYTFTDYYINWVKQRPGQGLEWIGWLFPGSGTTYYNEKFKGKATLTVAKSSSTAYMLLSSLTSEDSAVYFCARWGFGNYAFAYWGQGTLVTVSA

SEQ ID NO: 6 - protein, 107 aa, synthetic construct
DIVLTQSQKFMSATVGDRVSITCKASQNVGTAVAWYQQKPGQSPKLLIYSPSSRNTGVPDRFTGSGSGTDFTLTISSVQSEDLADYFCQQYSTYPRTFGGGTKLEIK

SEQ ID NO: 7 - protein, 116 aa, synthetic construct
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSA

SEQ ID NO: 8 - protein, 95 aa, synthetic construct
DIQMTQSPSSLSASVGDRVTITCRASQDISGYLNWLQQKPGGAIKRLIYTTSTLDSGVPKRFSGSGSGTDFATYYCLQYASSPFTFGGGTKVEIK

SEQ ID NO: 9 - protein, 8 aa, synthetic construct
GYTFTSYG

SEQ ID NO: 10 - protein, 8 aa, synthetic construct
IFPRSGNS

SEQ ID NO: 11 - protein, 14 aa, synthetic construct
ARHYYSSLYYAMDY

SEQ ID NO: 12 - protein, 6 aa, synthetic construct
ENLYGA

SEQ ID NO: 13 - skipped

SEQ ID NO: 14 - protein, 9 aa, synthetic construct
QNVLSTPRT

SEQ ID NO: 15 - protein, 5 aa, synthetic construct
DYYMY

SEQ ID NO: 16 - protein, 17 aa, synthetic construct
SISFDGTYTYYTDRVKG

SEQ ID NO: 17 - protein, 8 aa, synthetic construct
DRPAWFPY

SEQ ID NO: 18 - protein, 16 aa, synthetic construct
RSSQSIVHSNGNTYLE

SEQ ID NO: 19 - protein, 7 aa, synthetic construct
KVSNRFS

SEQ ID NO: 20 - protein, 9 aa, synthetic construct
FQGSHVPLT

SEQ ID NO: 21 - protein, 5 aa, synthetic construct
DYYIN

SEQ ID NO: 22 - protein, 17 aa, synthetic construct
WLFPGSGTTYYNEKFKG

SEQ ID NO: 23 - protein, 10 aa, synthetic construct
WGFGNYAFAY

SEQ ID NO: 24 - protein, 11 aa, synthetic construct
KASQNVGTAVA

SEQ ID NO: 25 - protein, 7 aa, synthetic construct
SPSSRNT

SEQ ID NO: 26 - protein, 9 aa, synthetic construct
QQYSTYPRT

SEQ ID NO: 27 - protein, 6 aa, synthetic construct
SSYAMS

SEQ ID NO: 28 - protein, 15 aa, synthetic construct
VIDSNGGSTYYPDTV

SEQ ID NO: 29 - protein, 8 aa, synthetic construct
SYTNLGAY

SEQ ID NO: 30 - protein, 11 aa, synthetic construct
RASQDISGYLN

SEQ ID NO: 31 - protein, 7 aa, synthetic construct
TTSTLDS

SEQ ID NO: 32 - protein, 9 aa, synthetic construct
LQYASSPFT

(19) United States
(12) Patent Application Publication
(10) Pub. No.: US 2024/0393338 A1
(43) Pub. Date: Nov. 28, 2024

(54) TIBTECH COMPOSITIONS AND METHODS FOR DETECTING CDH17

(71) Applicant: TIBERIAS TECHNOLOGY (HK) LIMITED, Shatin, N.T., (HK)

(72) Inventors: John Moonching LUK, Bellevue, WA (US); Stella SUN, Kennedy Town (HK); Pui Ling LEUNG, Pokfulam (HK)

(21) Appl. No.: 18/696,924

(22) PCT Filed: Oct. 8, 2022
(86) PCT No.: PCT/US2022/077828

§ 371 Date: Mar. 28, 2024

Related U.S. Application Data
(60) Provisional application No. 63/253,609, filed on Oct. 8, 2021.

(51) Int. Cl. G01N 33/574

(52) U.S. Cl. CPC G01N 33/57446; G01N 33/57492; G01N 2333/705; G01N 2474/20

(57) ABSTRACT

A method for detecting the amount of CDH17 protein in a sample from Automated Tissue Staining a subject, the method comprising: contacting the sample with a buffer at a temperature of at least 98° C. to provide a treated sample, contacting the treated sample with a capture antibody having a binding affinity to CDH17, wherein any exposed CDH17 expressing epitope in the treated sample is configured to bind to the capture antibody to provide a bound sample, contacting the bound sample with a detection molecule to provide a detection sample, wherein the detection molecule comprises a biocompatible enzyme conjugated to a secondary antibody having a binding affinity to the capture antibody, reacting the detection sample with a 3,3′Diamonobenzidine chromogen to provide an oxidized substrate, and determining the amount of CDH17 protein in the sample based on the amount of oxidized substrate.

### CROSS REFERENCE TO RELATED APPLICATIONS

This application claims the benefit of the filing date of U.S. Provisional Application Ser. No. 63/253,609 filed Oct. 8, 2021, under 35 U.S.C. 119(e), the entire disclosures of which are incorporated by reference herein.

### TECHNICAL FIELD

The present application relates to methods useful for the detection of cancer. In particular, the present application relates to automated, standardized, and high throughput Immunohistochemistry (IHC) method for detecting CDH17 expression in human tissue specimens, such as formalin-fixed paraffin-embedded (FFPE) tissue samples, tissue microarray (TMA), and frozen sections.

### BACKGROUND

Unless otherwise indicated herein, the materials described in this section are not prior art to the claims in this application and are not admitted being prior art by inclusion in this section.

Gastrointestinal (GI, i.e., esophagus, stomach, liver, bile duct/gall bladder, small intestine, pancreas and colorectum) cancers are a major cause of cancer death worldwide. An estimation of 4.8 million new cases and 3.4 million of cancer deaths in 2018 [1]. Esophagus, stomach, pancreas, and liver are the top five GI cancers that gave the worst prognosis among all primary cancer types [2].

Adenocarcinoma of an unknown primary origin is one of the most common clinical problems because metastatic adenocarcinomas from different locations may have a similar microscopic appearance making identification of their primary sites difficult. Immunohistochemical markers such as cytokeratin 7, cytokeratin 20, thyroid transcription factor 1, CDX2, prostate-specific antigen, and mesothelin are commonly used as histologic markers for GI cancer diagnosis. However, a low specificity of these markers resulting to an urgent need for a highly sensitive and more reliable diagnostic marker for adenocarcinomas of the digestive system [3, 4].

Cadherin-17 (CDH17 or CA17) is a biomarker for GI cancers characterized by its overexpression in stomach, liver, and colorectal cancers [3, 5, 6]. CDH17 has been reported to be a useful immunohistochemical marker for diagnosis, malignancy staging and prognosis [3, 5, 6]. Moreover, CDH17 is highly expressed in metastatic cancers, and the blockage of CDH17 expression and functions can markedly reduce lung metastasis of hepatocellular carcinoma (HCC) [7]. While CDH17 functions as a valid GI cancer diagnostic biomarker, an improvement in the detection strategy of CDH17 is essential to apply it for large scale screening approaches at a higher test sensitivity and specificity.

CDH17 has been shown to be involved in cancer progression and is associated with poor prognosis. Given CDH17 is expressed de novo or overexpressed at abnormal high levels in GI cancers including CRC, GE, and PDAC [8], determining the quantity of CDH17 expression in tissue samples may be a useful marker for diagnosis, differentiation of tissue origin, malignancy monitoring and disease prognosis.

While the treatment of GI cancer continues to rely heavily on conventional cytotoxic therapy, an increasing number of target agents are currently under development at Phase I/Il stages, such as the bi-specific TRAILR2/CDH17 antibody (BI 905711)[9] and the bi-specific T-cell engager CDH17/CD3 (ARB202) [10]. These treatments require companion diagnostic tests to define an appropriate population that will benefit most. CDH17 biomarker and the developed automated IHC assay could be used to provide information to a treating physician for selecting the eligible population for therapy. The increasing demand for immunohistochemistry in clinical diagnostics and drug companion diagnostics, in combination with an ongoing shortage of histology staffs, has brought forward the need for automation in immunohistochemistry.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides methods, agents, and compositions method for detecting CDH17 protein in a sample from a subject

In one embodiment, the application provides methods for detecting the amount of CDH17 protein in a sample from a subject. In one embodiment, the sample may be a formalin-fixed, paraffin-embedded (FFPE) or frozen cancer tissue. In one embodiment, the sample may be a gastrointestinal tissue. In one embodiment, the sample may be from a subject that is suspected of having a pre-cancerous condition or cancer. In one embodiment, the sample may be from a subject suffering from cancer. The method may be used to further characterize the cancer or pre-cancerous condition therefore provide guidance for treatment.

In one embodiment, the method includes first contacting the sample with a buffer at an elevated temperature to provide a treated sample. In one embodiment, the elevated temperature may be at least 80° C., 90° C., 98° C., 100° C., or 110° C. If the sample contains CDH17 protein, the step will provide a treated sample with exposed CDH17 expressing epitope.

The method then includes the step of contacting the treated sample with a capture antibody having a binding affinity to CDH17. Any exposed CDH17 expressing epitope present in the treated sample is configured to bind to the capture antibody to provide a bound sample. In one embodiment, the capture antibody may include an amino acid sequence having at least 75%, 80%, 85%, 90%, 95%, 98%, 99% sequence identity to SEQ ID NO 1, 2, 3, 4, 5, 6, 7, or 8. In one embodiment, the capture antibody may include 3 heavy chain CDRs having the SEQ ID NO: 9, 10, 11 and 3 light chain CDRs having the SEQ ID NO: 12, 13, 14. In one embodiment, the capture antibody may include 3 heavy chain CDRs having the SEQ ID NO: 15, 16, 17 and 3 light chain CDRs having the SEQ ID NO: 18, 19, 20. In one embodiment, the capture antibody may include 3 heavy chain CDRs having the SEQ ID NO: 21, 22, 23 and 3 light chain CDRs having the SEQ ID NO: 24, 25, 26. In one embodiment, the capture antibody may include 3 heavy chain CDRs having the SEQ ID NO: 27, 28, 29 and 3 light chain CDRs having the SEQ ID NO: 30, 31, 32.

The method further includes the step of contacting the bound sample with a detection molecule to provide a detection sample. In one embodiment, the detection molecule may include a biocompatible enzyme conjugated to a secondary antibody having a binding affinity to the capture antibody. In one embodiment, the bound sample and the detection molecule may be placed in contact in an automated staining instrument with controlled ambient temperature and humidity.

The method additionally includes the steps of reacting the detection sample with a 3,3′Diamonobenzidine chromogen to provide an oxidized substrate and determining the amount of CDH17 protein in the sample based on the amount of oxidized substrate. In one embodiment, a digital pathology system may be used to determine the amount of CDH17 protein in the sample based on the amount of oxidized substrate.

In one embodiment, the method may further include the step of scoring CDH17 expression in the sample and generating a data-based report.

In one embodiment, the method may further include quantifying the CDH17 protein expression in the sample, wherein different cell intensities are scored and combined to give a representative number.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows immunohistochemical staining pattern and intensity of CDH17 expression in paired normal (N) and tumor (T) colorectal specimens using five anti-CDH17 antibodies (Ab, 1 ug/mL), Lic3, 10C12, 7C5, 9A6, and 8G5, respectively;

FIG. 2 shows optimization of the primary anti-CDH17 antibody (Lic3) concentration to be used for the immunohistochemistry assay (IHC). Range of concentrations: 0 to 2 μg/ml;

FIG. 3 shows CDH17 (Lic3) immunoreactivity in normal human tissues. Only colon and small intestine showed positive membranous staining (original magnification: 10×);

FIG. 4 shows a) Representative pictures demonstrated strong CDH17 positive staining in GI neoplastic tissues including esophagus, stomach, colon, pancreas, gallbladder adenocarcinoma and cholangiocarcinoma but not in other neoplastic cancer tissue types (original magnification ×10); b) CDH17 staining intensity was quantified by M score showed >10 (as cut-off value) in all GI neoplastic tissues;

FIG. 5 shows CDH17 as a potential diagnostic marker for adenocarcinoma types of GI cancers. Automated CDH17 (Lic3) IHC assay and M Score (a digital scoring method) may be used to differentiate esophageal adenocarcinoma (EAC; n=35); intrahepatic cholangiocarcinoma (iCCA; n=25); pancreatic ductal adenocarcinoma (PDAC; n=37), gastric adenocarcinoma (GAC; n=40) and colorectal adenocarcinoma (CRC; n=112) from their healthy adjacent tissues (p<0.05);

FIG. 6 shows (a) Automated CDH17 IHC assay combined with the digital scoring method (M score) can differentiate GI tumor origin such as Intrahepatic Cholangiocarcinoma (iCAA) from Extrahepatic Cholangiocarcinoma (eCCA) and Esophageal Adenocarcinoma (EAC) from Esophageal Squamous Cell Carcinoma (ESCC); and (b) the corresponding IHC images of representative examples (original magnification 20×);

FIG. 7 shows CDH17 expression as quantified by M score is significantly associated with advanced stage of pancreatic ductal adenocarcinoma (PDAC);

FIG. 8 demonstrates high CDH17 expression is associated with poor patient prognosis. Colorectal cancer patients with differential CDH17 expression were stratified into two groups: CDH17 Low (M Score≤30) and CDH17 High (M Score>30). Patients with high level of CDH17 expression (M Score>30) are associated with a) Poorer overall survival (Log-rank p=0.021) and b) Higher Hazard Rate;

FIG. 9 shows an integrated CDH17 IHC assay platform comprising a unique brightfield imaging analysis system, a NMPA-approved high-throughput automated tissue staining, a digitalized sample scoring algorithm, and a cloud-based reporting system, which is catered for a variety of histopathology needs, including quantitative IHC scoring and whole slide imaging of IHC stained samples; and

FIG. 10 shows good correlation between automated and manual IHC scoring method. Percentage of positive cells scoring obtained by pathologist showed good agreement with the digital scoring method (M Score) by automated quantification: (a) by Pearson correlation analysis; and (b) by paired sample T-test.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present application is generally drawn, inter alia, to compositions, methods, apparatus, systems, devices, and/or computer program products related to immuno- and/or electro-chemical capacitors. The methods, devices, and systems disclosed herein may be implemented in any configuration for acquiring and processing data for the purposes of diagnosis or treatment of various gastrointestinal (GI) conditions described in this application, including but not limited to GI-cancer, CDH17-expressing normal and cancerous tissues, and may be executed utilizing a machine-readable medium for use in a computer or other electronic system embodying a set of instructions. Other features will be apparent from the accompanying drawings and from the detailed description that follows.

In some embodiments, the level of CDH17 expression in different types of diseases and malignancies can be ranked from low to high by using, without limitation, the cancer genome atlas (TCGA) RNA sequencing data (RNA Seq V2). In some embodiments, the high level of CDH17 expression is associated with GI cancers, including without limitation, colorectal, gastric, pancreatic, and esophageal cancer. The level of CDH17 expression is high in papillary renal cell carcinoma (PRCC), cholangiocarcinoma, and lung adenocarcinoma. In other embodiments, CDH17 antigen & mRNA restricted in small intestine and colon but no detectable in major organs like lung, heart, liver, and kidney.

The present application discloses the digital pathology system comprising immunohistochemical (IHC) assay for detecting the CDH17 expression in tissue specimens (or samples) with accuracy and consistency. In some embodiments, the immunohistochemical (IHC) assay for detecting the CDH17 expression in tissue specimens comprises a protocol and a kit. In some embodiments, the system comprises a brightfield imaging and computerized workflows with enhanced labor efficiency and data accessibility, a virtual slide reading option that streamlines communication between laboratories and pathologists and simplifies the sharing process.

In one embodiment, the application provides CDH17 (Lic3) IHC assay/method that is useful for the qualitative detection of the CDH17 protein in, for example, formalin-fixed, paraffin-embedded (FFPE) gastrointestinal tissue stained with an automated staining instrument. The assay/method disclosed herein may be used as an aid for diagnosis, monitoring, tumor origin differentiation, and prognosis. The application provides, among others, methods optimized to allow semi-quantification of CDH17 antigen expression in clinical tissues using Lic3 antibody, an automated IHC method, and/or digital pathology system.

In some embodiments, the assay disclosed herein are useful for diagnosis, malignancy monitoring, differentiation of tissue origin and disease prognosis. In some embodiments, the specimen may be cells, tissues, and biopsies frozen or embedded in, without limitation, agar block, formalin-fixed paraffin (FFPE), and tissue microarray (TMA). In some embodiments, the CDH17 assay kit of the method comprises a primary antibody selected from anti-CDH17 antibodies, a secondary antibody with binding affinity to the anti-CDH17 primary antibody and conjugated with a biocompatible enzyme, and a colorimetric substrate. In some embodiments, the IHC kit for detection of CDH17 expression in tissues consists of dilution buffer, wash buffer, antigen retrieval solution, blocker reagent, anti-CDH17 mouse monoclonal primary antibody, secondary antibody, and 3,3′-Diaminobenzidine (DAB) reagents.

The terms “a”, “an” and “the” as used herein are defined to mean “one or more” and include the plural unless the context is inappropriate.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, human and humanized antibodies as well as active fragments thereof.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against one antigenic site as a monoclonal monospecific antibody, or more than one antigenic site as a monoclonal multi-specific antibody. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any method. For example, the monoclonal antibodies to be used in accordance with the disclosure may be made by the hybridoma method first described by Kohler & Milstein, Nature, 256:495 (1975), or may be made by recombinant DNA methods (see, e.g., U.S. Pat. No. 4,816,567).

The disclosure of the present application may be understood more readily by reference to the following detailed description of specific embodiments and examples included herein.

Although the disclosure has been described with reference to specific details of certain embodiments thereof, it is not intended that such details should be regarded as limitations upon the scope of the disclosure. Indeed, various modifications of the disclosure in addition to those described herein will become apparent to those skilled in the art from the foregoing description and accompanying drawings. Such modifications are intended to fall within the scope of the appended claims.

### EXAMPLES

### Example 1. Anti-CDH17 Antibodies

Mouse anti-CDH17 antibodies have been developed using the hybridoma technology and their affinity for CDH17 has been characterized by binding affinity and epitope/domain mapping studies. The full-length sequences for the antibodies were listed in the present application and the CDR sequences were identified (underlined).

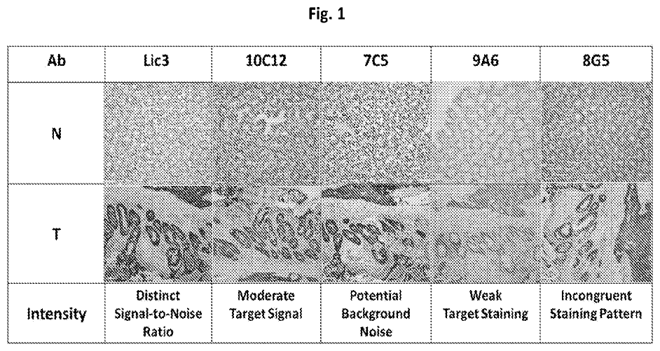

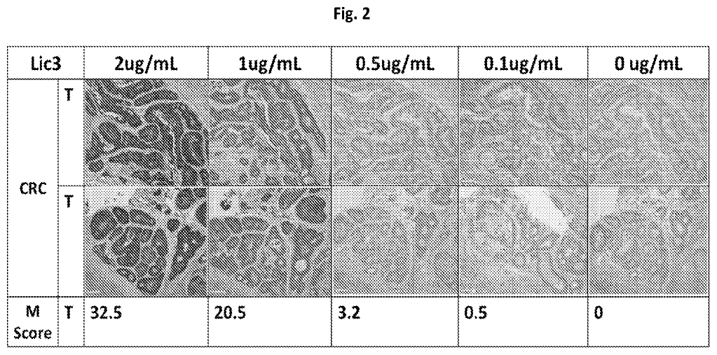

Among 10 CDH17 monoclonal antibodies screened for their ability to capture the CDH17 antigen from clinical specimens, 5 antibodies (Lic3, 10C12, 7C5, 9A6, and 8G5) were mapped to three domains of CDH17, respectively (Table 1). FIG. 1 shows the immunohistochemical staining pattern and intensity of CDH17 signals in paired normal (N) and tumor (T) colorectal specimens detected by using the five epitope-specific anti-CDH17 antibodies at the concentration of 1 ug/mL. Normal and tumor specimens (colon) were tested for the presence of CDH17 antigens captured by the primary anti-CDH17 antibodies in a semi-quantitative IHC format (FIG. 2). Cancer cell lines such as CDH17 positive CRC (DLD-1), CDH17 negative CRC (SW480) embedded in agar block were used as controls. Lic3 has been chosen among the primary antibodies that recognize various domains of CDH17, selection was guided by the highest sensitivity potential among over 200 specimens tested for the intended application. Lic3 was titrated to optimize contrast between positively staining tissue and non-specific background staining, with the highest primary antibody dilution. For the initial titration, an antibody concentration of 0 to 2 μg/mL was tested (CRC:Colorectal Cancer and T:Tumor). Quantification of Lic3 IHC staining using a digital scoring (M Score) method suggested that Lic3 primary antibody at concentration of 1 ug/mL is recommended for the CDH17 IHC assay.

### Example 2. Automated CDH17 IHC Assay

The method and diagnosis kit for detecting the expression of CDH17 protein may be used for both companion and standalone in vitro diagnostic purpose to analyze tissue/biopsy from different disease origins by IHC. Anti-CDH17 antibodies targeting different domains of the human CDH17 may be used to detect CDH17 protein at different sensitivity (Table 1). The IHC staining method using anti-CDH17 (Lic3) primary antibody with the detection kits may be used in automated tissue staining platforms for CDH17 antigen detection. In the automated platform, a unique brightfield imaging and capture system may generate digitized CDH17 positive signals on immuno-stained clinical tissues samples. An advanced digital pathology system capable of generating semi-quantitative report in cloud-based server for board-certified pathologist examination, may be integrated with the said method and diagnosis kit.

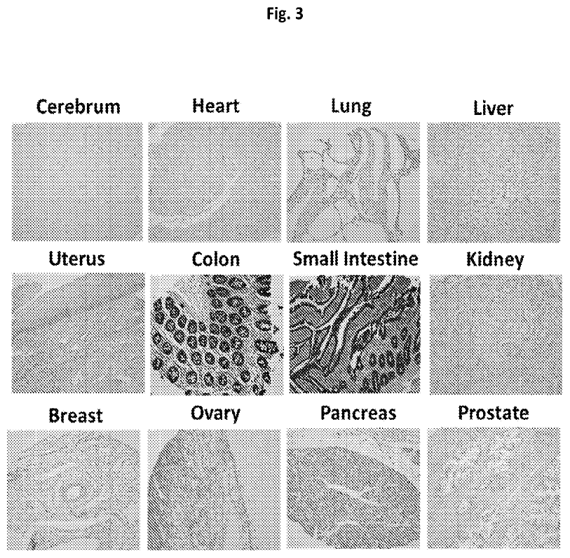

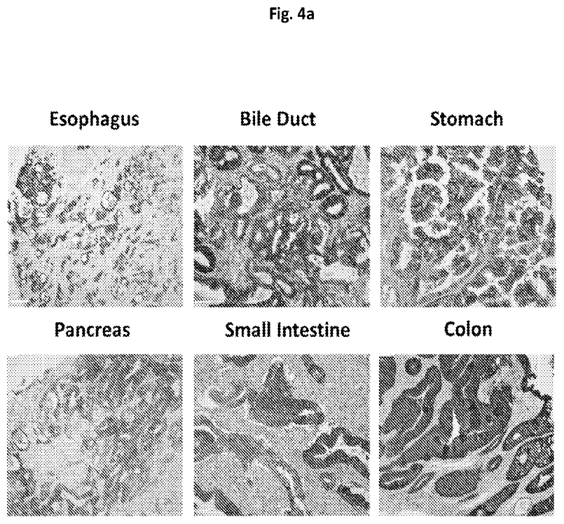

Steps towards assay optimization include determination of optimal antibody dilution, antigen retrieval conditions, and incubation time. Study was conducted to demonstrate specificity of the CDH17 (Lic3) IHC assay. One lot of anti-CDH17 (Lic3) was stained on commercially available tour of body (TOB) tissue microarrays (TMAs). Normal tissue screened including central nervous system, endocrine, breast, cardiovascular, gastrointestinal, genitourinary, only colon and small intestine showed positive CDH17 IHC staining (FIG. 3). The ideal Lic3 antibody dilution is 1 ug/mL that is the lowest antibody concentration that yields an optimal balance between sensitivity and specificity. A high pH-based commercial antigen retrieval solution was used to optimize primary antibody binding. The combination of staining conditions that produces an optimal signal-to-background ratio was adopted for the remainder of the validation process.

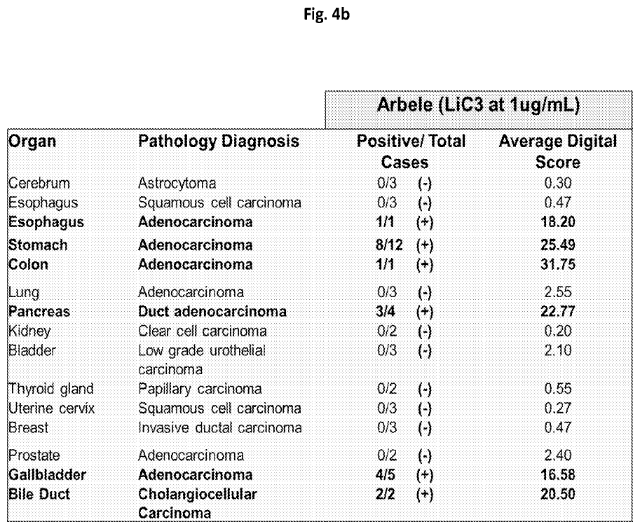

Assay interpretation criteria may be in line with those in the literatures and subjected to pathologists' validation. CDH17 antigen & mRNA restricted in small intestine and colon but no detectable in major organs like lung, heart, liver, and kidney (FIG. 3). On the other hand, gastrointestinal cancer, such as esophagus, stomach, colon, pancreas, gallbladder adenocarcinoma and cholangiocarcinoma, but not other neoplastic tissues, showed strong positive staining of CDH17 (Table 2, FIG. 4a).

Of the automated IHC system, quantitative digital scoring method gave a high average scoring (>10) among GI cancer when comparing to other non-GI caner types; and representative images of GI tumor tissue demonstrated strong intratumoral CDH17 signal intensity. Indeed, interpretation was carried out by comparing the pathologist manual scoring to the digital scoring, which revealed a consistent correlation between both methods (Table 3, FIG. 4b).

### Example 3. Operating Protocol

While automated staining system may be further optimized, the routine IHC operating procedures were set and listed below:
1. For the IHC assay, FFPE tissue sections are placed in high pH buffer and inserted into a semi-automated antigen retrieval system;
2. Preparation of the reagents (blocking, primary antibody, secondary antibody, etc.); CDH17 epitope retrieved samples were placed into the automated stainer for IHC process;
3. Operation of the automated stainer according to the assigned software; primary antibody and the secondary antibody to be dispensed onto the sample. Incubation times for other reagents may be varied.
4. IHC assay is carried out in a maintained environment, and slides are removed from the stainer and prepared for counterstaining with Hematoxylin along with mounting of slides;
6. CDH17 expression in the tissue section is quantified using a digital image analysis software;
7. Assay scoring algorithm is used to assist in the interpretation of the assay results.

Validation samples: Cancer cell lines with CDH17 positive and negative expression were embedded in agar block and used for validation. Normal and patients' tissue specimens from different organs were used to demonstrate an appropriate spectrum of staining within a particular tumor type or across different tumor types. In this context, CDH17 protein expression are restricted to GI specimens by in house CDH17 IHC assay.

### Example 4. M Score of Automated CDH17 IHC Assay

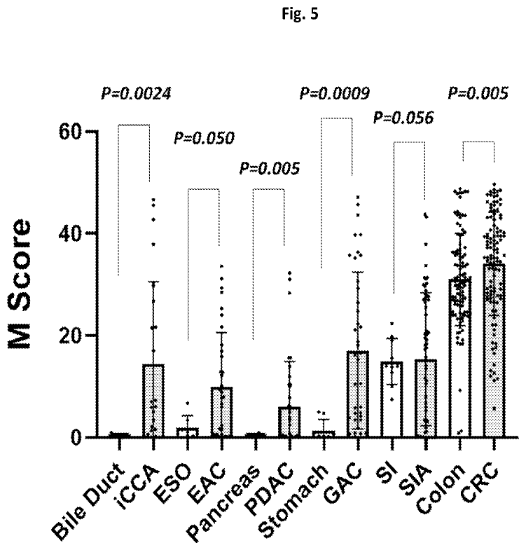

Automated CDH17 IHC assay confirmed that CDH17 is a useful diagnostic marker for adenocarcinoma types of GI cancers. Results of CDH17 expression may be quantified by using the digital scoring method, M Score. FIG. 5 shows that M Score may be used to differentiate esophageal adenocarcinoma (EAC; n=35); intrahepatic cholangiocarcinoma (iCCA; n=25); pancreatic ductal adenocarcinoma (PDAC; n=37), gastric adenocarcinoma (GAC; n=40) and colorectal adenocarcinoma (CRC; n=112) from their healthy adjacent tissues (p<0.05).

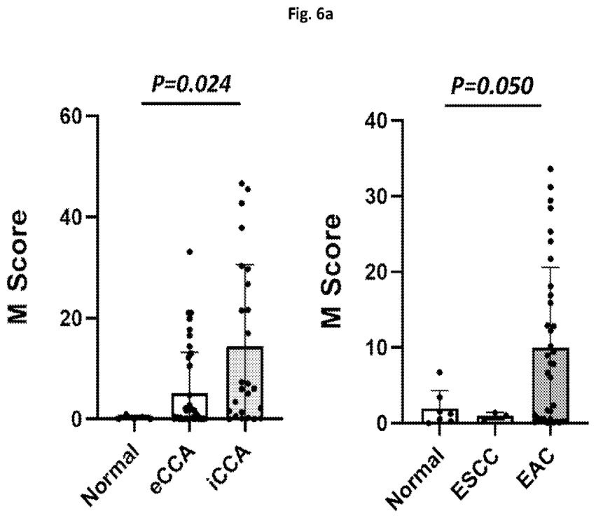

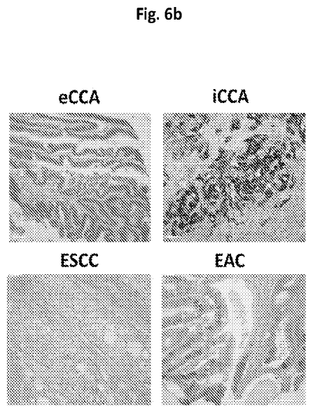

Automated CDH17 IHC assay may be used to differentiate the origin of GI cancer. as measured by M Score, CDH17 (Lic3) IHC assay can be used to significantly differentiate subtypes of intrahepatic cholangiocarcinoma (iCCA) and extrahepatic cholangiocarcinoma (eCCA) with p=0.0024, as well as esophagus cancer, such as esophageal squamous cell carcinoma (ESCC) and esophageal adenocarcinoma (EAC) with p=0.050 (FIG. 6).

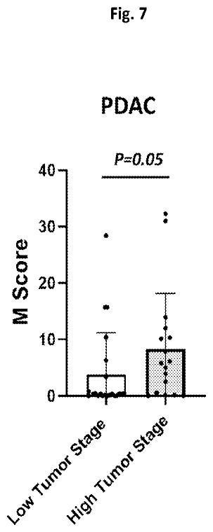

Automated CDH17 IHC assay may be used to detect pancreatic ductal adenocarcinoma (PDAC) at advanced stage by measuring M score corresponding to the level of CDH17 expression (FIG. 7).

### Example 5. M Score Cutoff Value for Prognosis

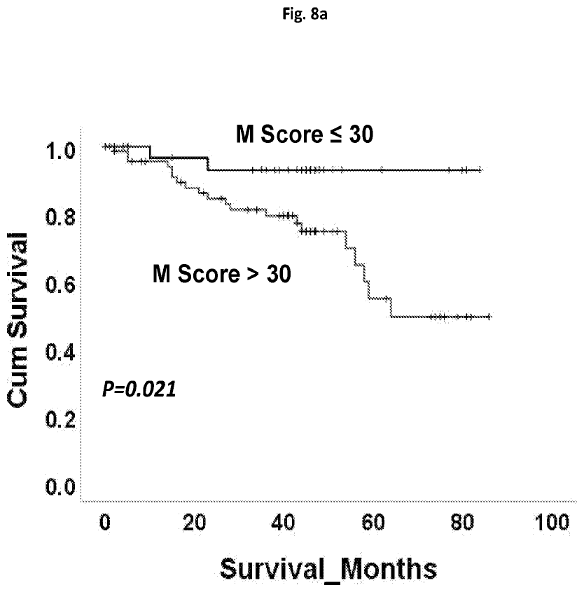

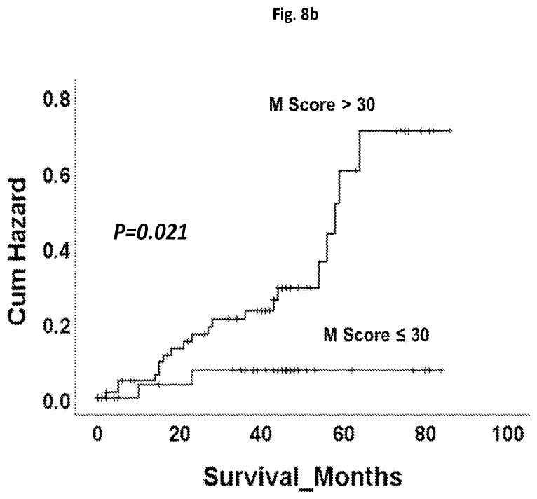

The automated therapy-guiding IHC assay may be desired to be integrated as a standardized reporting template into the cloud-based reporting. The M-score is a quantitative measure of membrane staining with a calculated range of 0 to 50. FIG. 8 shows the stratification for M Score 30 and M Score>30 in all patients with colorectal cancer, indicating that patients with a higher positive rate of CDH17 (M Score−cut-off value>30) are associated with poorer overall survival (Log-rank p=0.021) and higher hazard rate. Therefore, high levels of CDH17 expression are associated with poor patient prognosis.

### Example 6. Automated and Integrated CDH17 IHC System

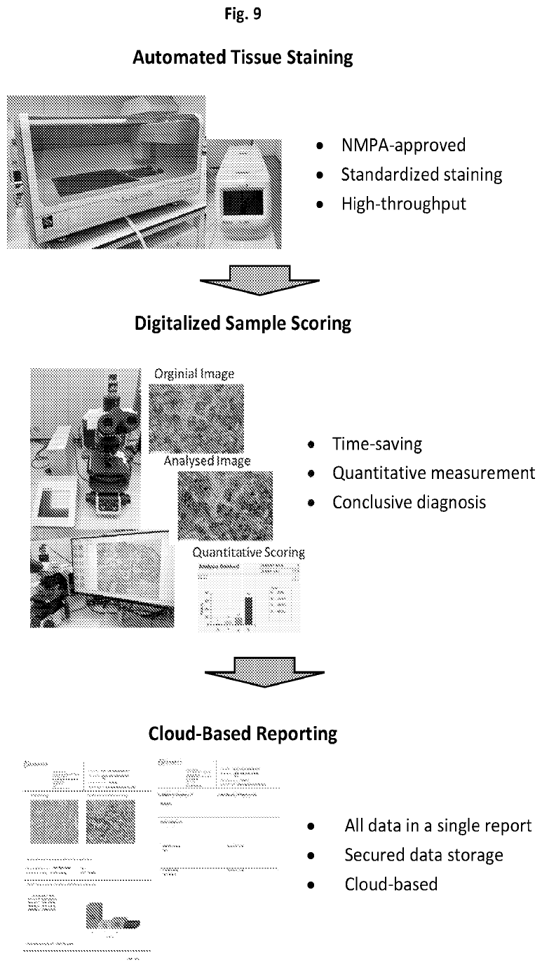

A brightfield imaging analysis system was integrated into the automation of CDH17 IHC assay platform. As illustrated in FIG. 9, the integrated CDH17 IHC system also includes NMPA (the National Medical Products Administration in China)—approved high-throughput automated tissue staining, digitalized sample scoring algorithm and cloud-based reporting system. This brightfield imaging-analysis system was catered for a variety of histopathology needs, such as quantitative IHC Scoring and Whole Slide Imaging of H&E (Hematoxylin and eosin) stains/IHC samples.

Quality Control: CDH17 stability and reproducibility was tested as follows: from each of five FFPE blocks of tumor tissue with retained expression, 10 sections were cut at the beginning of a consecutive 10-days validation period. Analysis was performed each day on a single unstained FFPE slide from each of those five blocks on a rotating basis by two different histotechnologists. Assay repeatability was tested as follows: from each of three FFPE blocks of tumor tissues with retained expression, three sections were stained on two different machines. Each of the assays showed consistent performance across these validation experiments.

### Example 7. Automated CDH17 IHC Assay as a Companion/Standalone Diagnostic Test

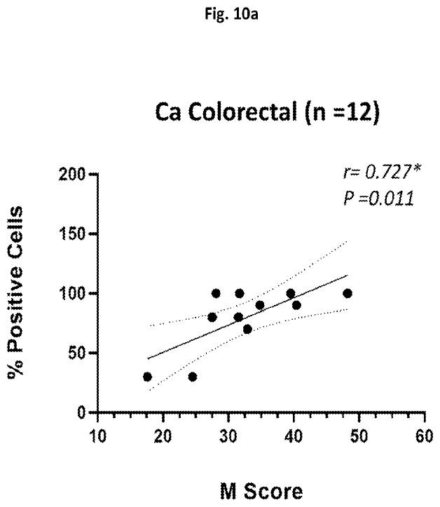

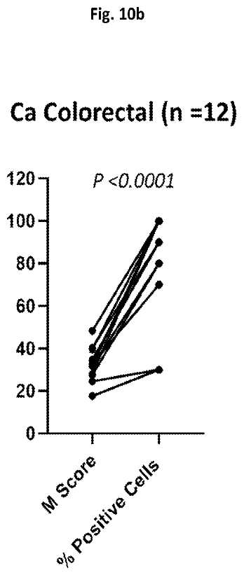

The use of a highly specific CDH17 antibody that binds CDH17 in tissue samples would be useful to assist diagnosis, disease staging, tumor origin differentiation, and prognosis for multiple advanced malignancies of the gastrointestinal tract including cholangiocarcinoma, esophageal, pancreatic, gastric, and colorectal adenocarcinomas. In all these malignancies, traditional pathology/digital scoring (IHC membrane scoring method) may be used for CDH17 quantification. A good correlation showed between traditional IHC scoring and digital method (FIG. 10). A digital score (such as M Score) generated by the digital pathology system would be indicative for disease assessment. In research and development targets, this cutoff may be used as an early indicator of disease. Differentiation and staging of different malignancy subtypes for each tissue may be made at a higher threshold of the score. The high specificity and sensitivity of the tissue diagnostic test may be used confidently as both companion diagnostic tool in clinical studies and a standalone IVD assay.

Traditional manual methods of the immunohistochemical staining require copious amounts of time in addition to the introduction of human and technical variations. A standardization of the process in its entirety is nearly unachievable. In contrast, the automated technique of IHC staining allows ease of use with controlled temperature and humidity settings, minimizing environmental effects while ensuring precise timings in the different steps of the staining process. Exact reagent amounts needed for each stain run would be listed, ensuring proper reagent dispensing. Optimization and validation of the automated technique has been performed by precision studies that consist of intraday, inter-day, and inter-platform comparisons which serve to increase the confidence of the assay results. Verification of staining results in different tissue samples in the various samples provided by tissue specific and multiple organ Tissue Micoarray Assays (TMAs) were done by comparing with known and tested staining.

Clinical application of CDH17 IHC assay: the assay is intended to be used for diagnosis, malignancy monitoring, differentiation of tissue origin, disease prognosis and companion diagnostics.

### REFERENCES

1. Arnold, M., et al., Global Burden of 5 Major Types of Gastrointestinal Cancer. Gastroenterology, 2020, 159(1): p. 335-349 e15.
2. Arnold, M., et al., Progress in cancer survival, mortality, and incidence in seven high-income countries 1995-2014 (ICBP SURVMARK-2): a population-based study. Lancet Oncol, 2019, 20(11): p. 1493-1505.
3. Su, M. C., et al., Cadherin-17 is a useful diagnostic marker for adenocarcinomas of the digestive system. Mod Pathol, 2008, 21(11): p. 1379-86.
4. Weimann, A., et al., CDX2 and LI-cadherin expression in esophageal mucosa: use of both markers can facilitate the histologic diagnosis of Barrett's esophagus and carcinoma. Int J Surg Pathol, 2010, 18(5): p. 330-7.
5. Panarelli, N.C., et al., Tissue-specific cadherin CDH17 is a useful marker of gastrointestinal adenocarcinomas with higher sensitivity than CDX2. Am J Clin Pathol, 2012, 138(2): p. 211-22.
6. Suh, Y. S., et al., The combined expression of metaplasia biomarkers predicts the prognosis of gastric cancer. Ann Surg Oncol, 2012, 19(4): p. 1240-9.
7. Lee, N. P., et al., Role of cadherin-17 in oncogenesis and potential therapeutic implications in hepatocellular carcinoma. Biochim Biophys Acta, 2010, 1806(2): p. 138-45.
8. Vafa, O. and N. D. Trinklein, Perspective: Designing T-Cell Engagers With Better Therapeutic Windows. Front Oncol, 2020, 10: p. 446.
9. Garcia-Martinez, J. M., et al., Selective Tumor Cell Apoptosis and Tumor Regression in CDH17-Positive Colorectal Cancer Models using BI 905711, a Novel Liver-Sparing TRAILR2 Agonist. Mol Cancer Ther, 2021, 20(1): p. 96-108.
10. Wong, D. A., et al., Efficacy and preclinical safety of ARB202, a potential first-in-class anti-CDH17/CD3 bispecific T-cell engager, for treatment of pancreatic and colorectal cancers. Journal of Clinical Oncology, 2021, 39(3_suppl): p. 405-405.

### TABLES

TABLE 1
The signal-to-noise ratio by various
anti-CDH17 primary antibodies in IHC assay.

| Clone | Host | Isotype | Domain | Dilution | Staining Results |
|---|---|---|---|---|---|
| LiC3 | Mouse | IgG2a | 2 | 1:100 | Distinct signal-to- |
| | | | | | noise ratio |
| 10C12 | Mouse | IgG2a | 1 | 1:100 | Moderate target staining |
| 7C5 | Mouse | IgG2a | 1 | 1:100 | High background noise |
| 9A6 | Mouse | IgG2a | 4 | 1:100 | Weak target staining |
| 8G5 | Mouse | IgG1 | 1 | 1:100 | Incongruent |
| | | | | | staining pattern |

TABLE 2
High CDH17 expression in all types of GI cancer.

| | Tissue | No. of | Positive | % |
|---|---|---|---|---|
| Cancer Type | Type | Samples | Number | Positive |
| Colon | FFPE | 111 | 110 | 99% |
| Esophagus | | | | |
| ESCC | TMA | 3 | 0 | 0% |
| EAC | | 35 | 20 | 57% |
| Gastric (GAC) | TMA | 40 | 25 | 63% |
| Pancreas (PDAC) | TMA | 37 | 15 | 41% |
| Small Intestine (SIA) | TMA | 60 | 40 | 67% |
| Bile Duct | | | | |
| iCCA | TMA | 22 | 12 | 55% |
| eCCA | | 48 | 6 | 12.5% |

TABLE 3
Immunoreactivity in human normal tissues

| | # positive/ | M score |
|---|---|---|
| Organ | total cases | (Average) |
| Cerebrum | 0/2 | 0.0 |
| Heart | 0/2 | 0.2 |
| Lung | 0/2 | 0.1 |
| Liver | 0/2 | 0.2 |
| Uterus | 0/2 | 0.5 |
| Colon | 2/2 | 20.5 |
| Small intestine | 10/10 | 29.9 |
| Kidney | 0/2 | 0.0 |
| Breast | 0/2 | 0.1 |
| Ovary | 0/2 | 0.6 |
| Pancreas | 0/2 | 0.0 |
| Prostate | 0/2 | 0.4 |

SEQUENCE LISTING
>Seq ID NO 1: m7C5 VH
QVQLQQSGAELARPGASVKLSCKASGYTFTSYGLSW
VKQRTGQGLEWIGEIFPRSGNSYYNEKFKGKAALTA
DKSSSTAYMQLSSLTSEDSAVYFCARHYYSSLYYAM
DYWGQGTSVTVSS
>Seq ID NO 2: m7C5 VL
DIQVTQSPASLSASVGESVSITCGTNENLYGALNWY
QRKQGKSPQLLIYGATNLADGMSSRFSGSGSGRQYS
LKISSLHPDDVATYYCQNVLSTPRTFGGGTKLEIK
>Seq ID NO 3: mLic3 VH
EVQLVESGGGLVKPGGSLKLSCAASGFSFSDYYMYW
VRQAPEKRLEWVASISFDGTYTYYTDRVKGRFTISR
DNAKNNLYLQMSSLKSEDTAMYYCARDRPAWFPYWG
QGTLVTVSA
>Seq ID NO 4: mLic3 VL
DVLMTQIPLSLTVSLGDQASISCRSSQSIVHSNGNT
YLEWYLQRPGQSPKLLIYKVSNRFSGVPDRFSGSGS
GTDFTLKISRVEAEDLGVYYCFQGSHVPLTFGAGTK
LELK
Seq ID NO 5: m9A6 VH
EVKLQESGPELVKPGASVTISCKASGYTFTDYYINW
VKQRPGQGLEWIGWLFPGSGTTYYNEKFKGKATLTV
AKSSSTAYMLLSSLTSEDSAVYFCARWGFGNYAFAY
WGQGTLVTVSA
Seq ID NO 6: m9A6 VL
DIVLTQSQKFMSATVGDRVSITCKASQNVGTAVAWY
QQKPGQSPKLLIYSPSSRNTGVPDRFTGSGSGTDFT
LTISSVQSEDLADYFCQQYSTYPRTFGGGTKLEIK
>Seq ID NO 7: m10C12 VH
EVQLVESGGGLVQPGGSLRLSCAASGFTFSSYAMSW
VRQTPGKGLEWVAVIDSNGGSTYYPDTVKDRFTISR
DNSKNTLYLQMNSLRAEDTAVYYCSSYTNLGAYWGQ
GTLVTVSA
>Seq ID NO 8: m10C12 VL
DIQMTQSPSSLSASVGDRVTITCRASQDISGYLNWL
QQKPGGAIKRLIYTTSTLDSGVPKRFSGSGSGTDFA
TYYCLQYASSPFTFGGGTKVEIK
>SEQ ID NO 9: m7C5 heavy chain CDR-H1 sequence
GYTFTSYG
>SEQ ID NO 10: m7C5 heavy chain CDR-H2 sequence
IFPRSGNS
>SEQ ID NO 11: m7C5 heavy chain CDR-H3 sequence
ARHYYSSLYYAMDY
>SEQ ID NO 12: m7C5 light chain CDR-L1 sequence
ENLYGA
>SEQ ID NO 13: m7C5 light chain CDR-L2 sequence
GAT
>SEQ ID NO 14: m7C5 light chain CDR-L3 sequence
QNVLSTPRT
>SEQ ID NO 15: mLic3 heavy chain CDR-H1 sequence
DYYMY
>SEQ ID NO 16: mLic3 heavy chain CDR-H2 sequence
SISFDGTYTYYTDRVKG
>SEQ ID NO 17: mLic3 heavy chain CDR-H3 sequence
DRPAWFPY
>SEQ ID NO 18: mLic3 light chain CDR-L1 sequence
RSSQSIVHSNGNTYLE
>SEQ ID NO 19: mLic3 light chain CDR-L2 sequence
KVSNRFS
>SEQ ID NO 20: mLic3 light chain CDR-L3 sequence
FQGSHVPLT
>SEQ ID NO 21: m9A6 heavy chain CDR-H1 sequence
DYYIN
>SEQ ID NO 22: m9A6 heavy chain CDR-H2 sequence
WLFPGSGTTYYNEKFKG
>SEQ ID NO 23: m9A6 heavy chain CDR-H3 sequence
WGFGNYAFAY
>SEQ ID NO 24: m9A6 light chain CDR-L1 sequence
KASQNVGTAVA
>SEQ ID NO 25: m9A6 light chain CDR-L2 sequence
SPSSRNT
>SEQ ID NO 26: m9A6 light chain CDR-L3 sequence
QQYSTYPRT
>SEQ ID NO 27: m10C12 heavy chain CDR-H1 sequence
SSYAMS
>SEQ ID NO 28: m10C12 heavy chain CDR-H2 sequence
VIDSNGGSTYYPDTV
>SEQ ID NO 29: m10C12 heavy chain CDR-H3 sequence
SYTNLGAY
>SEQ ID NO 30: m10C12 light chain CDR-L1 sequence
RASQDISGYLN
>SEQ ID NO 31: m10C12 light chain CDR-L2 sequence
TTSTLDS
>SEQ ID NO 32: m10C12 light chain CDR-L3 sequence
LQYASSPFT

What is claimed is:

1. A method for detecting the amount of CDH17 protein in a sample from a subject, the method comprising:
contacting the sample with a buffer at a temperature of at least 98° C. to provide a treated sample,
contacting the treated sample with a capture antibody having a binding affinity to CDH17, wherein any exposed CDH17 expressing epitope in the treated sample is configured to bind to the capture antibody to provide a bound sample,
contacting the bound sample with a detection molecule to provide a detection sample, wherein the detection molecule comprises a biocompatible enzyme conjugated to a secondary antibody having a binding affinity to the capture antibody,
reacting the detection sample with a 3,3′Diamonobenzidine chromogen to provide an oxidized substrate, and
determining the amount of CDH17 protein in the sample based on the amount of oxidized substrate.

2. The method of claim 1, further comprising scoring CDH17 expression in the sample and generating a data-based report.

3. The method of claim 1, further comprising quantifying the CDH17 protein expression in the sample, wherein different cell intensities are scored and combined to give a representative number.

4. The method of claim 1, wherein contacting the bound sample with a detection molecule comprises contacting the bound sample with the detection molecule in an automated staining instrument with controlled ambient temperature and humidity.

5. The method of claim 1, wherein determining the amount of CDH17 protein in the sample comprises determining the amount of CDH17 protein in the sample based on the amount of oxidized substrate using a digital pathology system.

6. The method of claim 1, wherein the capture antibody comprises an amino acid sequence having at least 98% sequence identity to SEQ ID NO 1, 2, 3, 4, 5, 6, 7, or 8.

7. The method of claim 1, wherein the capture antibody comprises:
3 heavy chain CDRs having the SEQ ID NO: 9, 10, 11 and 3 light chain CDRs having the SEQ ID NO: 12, 13, 14,
3 heavy chain CDRs having the SEQ ID NO: 15, 16, 17 and 3 light chain CDRs having the SEQ ID NO: 18, 19, 20,
3 heavy chain CDRs having the SEQ ID NO: 21, 22, 23 and 3 light chain CDRs having the SEQ ID NO: 24, 25, 26, or
3 heavy chain CDRs having the SEQ ID NO: 27, 28, 29 and 3 light chain CDRs having the SEQ ID NO: 30, 31, 32.

8. The method of claim 1, wherein the sample comprises a formalin-fixed, paraffin-embedded (FFPE) or frozen gastrointestinal cancer tissue.
